(19) United States
(12) Patent Application Publication
(10) Pub. No.: US 2013/0096433 A1
(43) Pub. Date: Apr. 18, 2013

(54) System and Method for Unattended Monitoring of Blood Flow

(71) Applicants: Sonetics Ultrasound, Inc., Ann Arbor, MI (US); The Regents of the University of Michigan, Ann Arbor, MI (US)

(72) Inventors: David Lemmerhirt, Ann Arbor, MI (US); Xiaoyang Cheng, Ann Arbor, MI (US); William F. Weitzel, Ypsilanti, MI (US); Grant H. Kruger, Ann Arbor, MI (US); Collin A. Rich, Ypsilanti, MI (US)

(73) Assignees: THE REGENTS OF THE UNIVERSITY OF MICHIGAN, Ann Arbor, MI (US); SONETICS ULTRASOUND, INC., Ann Arbor, MI (US)

(21) Appl. No.: 13/655,191

(22) Filed: Oct. 18, 2012

Related U.S. Application Data
(60) Provisional application No. 61/548,677, filed on Oct. 18, 2011.

(51) Int. Cl. A61B 8/06; A61B 8/14

(52) U.S. Cl. 600/441; 600/454

(57) ABSTRACT

A system and method for unattended monitoring of blood flow using an array of transmitter elements configured to transmit acoustic signals along a transmission direction; an array of receiver elements configured to receive acoustic signals originating from the array of transmitter elements, wherein the array of transmitter elements is arranged approximately orthogonal to the array of receiver elements and wherein each receiver element is configured to provide an output signal; an electronics system comprising a transmitter control subsystem configured to adjust the transmission direction, a receiver control subsystem configured to selectively activate and deactivate receiver elements, thus defining an acoustic aperture, an analog adder circuit, and signal processing circuitry; a fastener to position the elements on a patient; and a processor in communication with the electronics system and configured to enable self-alignment of transmitted acoustic signals based on received acoustic signals and determine a blood flow parameter.

### CROSS-REFERENCE TO RELATED APPLICATIONS

This application claims the benefit of U.S. Provisional Application No. 61/548,677 filed 18 Oct. 2011, titled “Device and Method for Monitoring Blood Flow”, which is incorporated in its entirety by this reference.

### TECHNICAL FIELD

This invention relates generally to the fluid flow measurement field, and more specifically to an improved system and method for unattended monitoring of blood flow.

### BACKGROUND

There are many applications, particularly in medical care, in which accurate measurement and other monitoring of blood flow in blood vessels is important. For instance, velocity measurements of blood flow may enable assessment of functionality of the cardiovascular system (e.g., such as monitoring cardiac performance or the condition of blood vessels). Conventional blood flow measurement and monitoring devices that utilize ultrasound technology are able to measure blood flow velocity sending an acoustic pulse or sequence of acoustic pulses into the body, and analyzing the reflected acoustic pulses. Various characteristics such as the angle of insonification relative to the measured flow, and location of ultrasound transmitters and receivers, affect the measurable field, accuracy, and/or precision of the flow monitoring. However, these conventional devices are relatively inflexible in use. For example, arrays of ultrasound transmitters and receivers must be placed in a very specific location, such that control over the location and angle of insonification requires physical movement of the transmitters and receivers.

Thus, there is a need in the fluid flow measurement field to create an improved system and method for unattended monitoring of blood flow. This invention provides such an improved system and method for unattended monitoring of blood flow.

### BRIEF DESCRIPTION OF THE FIGURES

FIG. 1A is a block diagram of the components of the system for monitoring blood flow of the preferred embodiment;

FIG. 1B is a schematic of a portion of the system with a blood vessel;

FIGS. 2A and 2B are exemplary schematics of a set of active receiver elements defining an acoustic aperture and a split acoustic aperture, respectively, in the device for monitoring fluid flow of a preferred embodiment;

FIGS. 3 and 4 are cross-sectional and top view schematics, respectively, of an integrated circuit/transducer device in the device for monitoring fluid flow of a preferred embodiment;

FIG. 5A is a schematic of the electronics system in the system for monitoring blood flow of a preferred embodiment;

FIGS. 5B and 5C depict example embodiments of a fastener;

FIGS. 6A-6D are schematics of a method for monitoring blood flow of a preferred embodiment;

FIG. 7A is a flowchart of a method for unattended monitoring of blood flow;

FIG. 7B is a flowchart of a method for processing acoustic signals to determine a fluid flow parameter;

FIG. 8A is a schematic of adjustment or steering of the insonification angle in the device and method for monitoring blood flow of a preferred embodiment;

FIGS. 8B and 8C are schematics of adjustment of the acoustic aperture location and size, respectively, in the device and method for monitoring blood flow of a preferred embodiment; and

FIG. 9 is a schematic of a method for estimating volumetric flow rate of blood flow.

### DESCRIPTION OF THE EMBODIMENTS

The following description of embodiments of the invention is not intended to limit the invention to these embodiments, but rather to enable any person skilled in the art to make and use this invention.

### System for Unattended Monitoring of Blood Flow

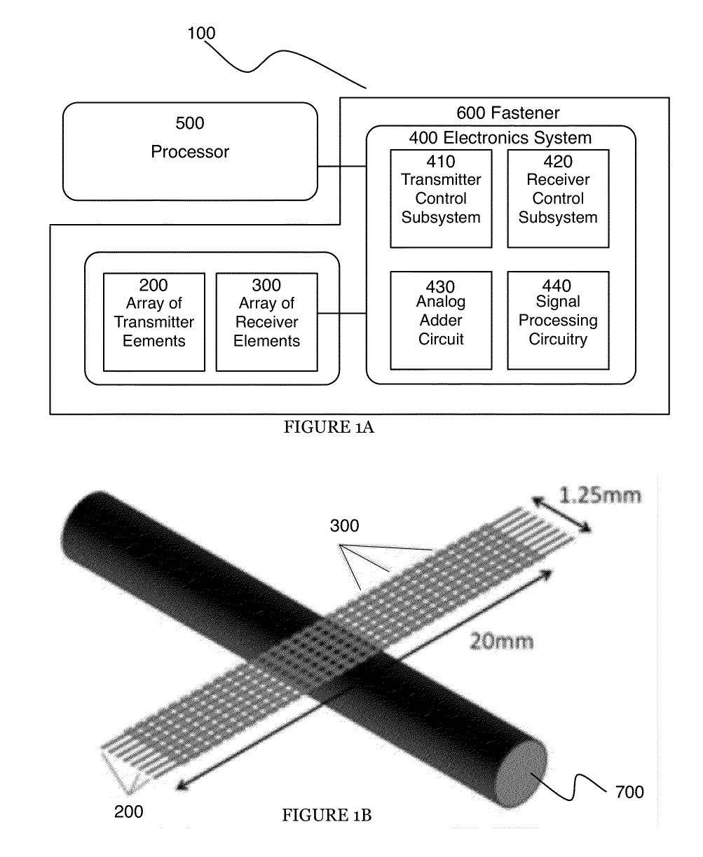

As shown in FIGS. 1, 2, and 5, the system 100 of a preferred embodiment comprises: an array of transmitter elements 200 configured to transmit acoustic signals 250 along a transmission direction 255; an array of receiver elements 300 configured to receive acoustic signals 250 originating from the array of transmitter elements 200, wherein the array of transmitter elements 200 is arranged approximately orthogonal to the array of receiver elements 200; an electronics system 400 comprising a transmitter control subsystem 410 configured to adjust the transmission direction, a receiver control subsystem 420 configured to selectively activate and deactivate receiver elements, thus defining an acoustic aperture 320 characterized by activated receiver elements 310; signal processing circuitry configured to condition the acoustic signals received by the activated receiver elements 310; fastener 600 to position the array of transmitter elements 200, the array of receiver elements 300, and the electronics system 400 on a patient; and a processor 500 in communication with the electronics system 400 and configured to provide feedback to the electronics system to enable self-alignment of transmitted acoustic signals based on received acoustic signals, thereby providing unattended monitoring of blood flow, and determine a fluid flow parameter based on Doppler flow information extracted from acoustic signals 250 received by the activated receiver elements 310. In one variation the electronics system 400 preferably further includes an analog adder circuit 430 configured to produce a combined output 350 by combining output signals provided by the activated receiver elements 310, and signal processing circuitry 440 configured to condition the combined output 350. The system 100 is preferably configured to provide an acoustic aperture 320 that is adjustable in size (corresponding to the number of active receiver elements) and/or position (corresponding to the location of the active receiver elements), such that the geometry of the beam of received acoustic signals 250 can be customized depending on the application (e.g. fluid vessel size or fluid vessel depth) or particular monitoring context. In the preferred embodiment, the system 100 is used to measure blood flow such as in blood vessels in a human or animal.

The array of transmitter elements 200 functions to transmit acoustic signals 250 along a transmission direction 255, wherein the acoustic signals 250 are reflected and received by the array of receiver elements 300. In one embodiment, the array of transmitter elements 200 comprises piezoelectric transducer elements, wherein applied electrical pulses are converted to mechanical vibrations that are transmitted to a surrounding medium by the piezoelectric transmitters. In another embodiment, the array of transmitter elements 200 comprises capacitive micromachined ultrasonic transducer (CMUT) elements, which generate vibrations in a surrounding medium in response to being subjected to an applied AC signal.

In the embodiment of the array of transmitter elements 200 that includes piezoelectric transmitter elements, individual piezoelectric transmitters are coupled to form an array that can be positioned to monitor blood flow in a blood vessel. Application of an AC signal induces cyclic polarization of molecules in the transmitter material, which results in oscillations that produce acoustic vibrations in a surrounding medium. The piezoelectric transmitter material may be natural or synthetic. The piezoelectric transmitter elements may further be coupled to acoustic lenses that function to focus emitted acoustic signals.

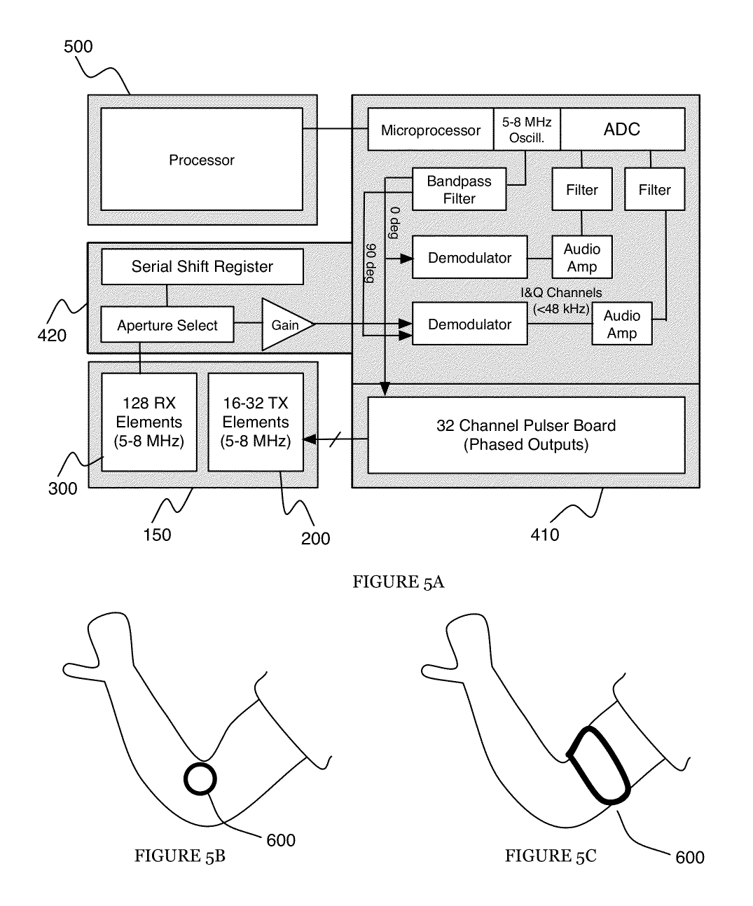

In the embodiment of the array of transmitter elements 200 that includes CMUT transmitter elements, as shown in FIG. 4, the array of transmitter elements 200 is defined by wiring together electrodes located on plates of individual CMUT units 150. An applied radio frequency (RF) voltage waveform causes the plates to vibrate due to storage of elastic energy and release of kinetic energy, which generates an acoustic signal 250 in a surrounding medium. In one variation, the RF voltage waveform is added to a constant direct current (DC) baseline voltage. Preferably, the plates on which the array of transmitter elements is located are composed of an insulating material, such as a dielectric material, coupled to a metal electrode. Alternatively, the plates are entirely composed of a conductive material or semiconductor.

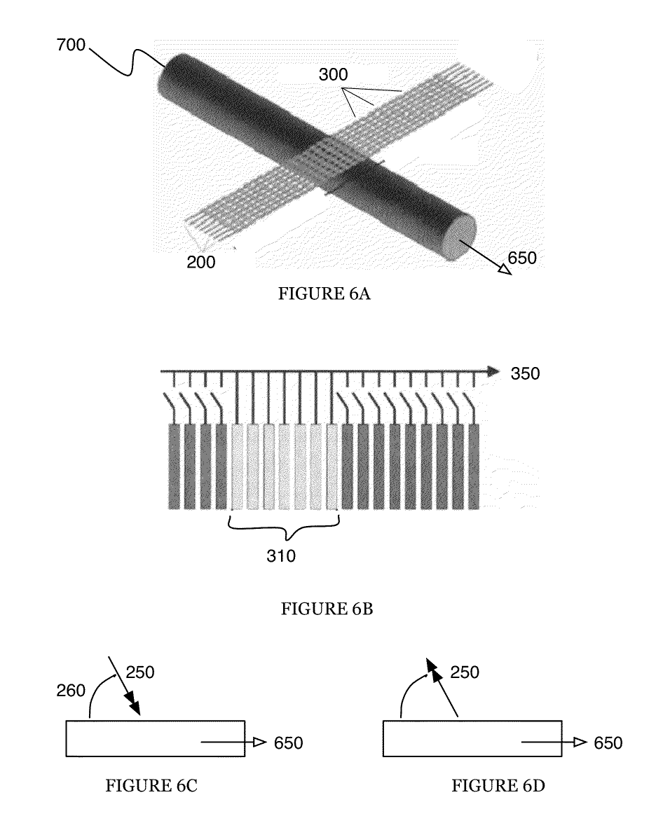

In the preferred embodiment, the array of transmitter elements 200 is arranged along an axis parallel to the long dimension of a fluid vessel 600 being monitored, so that the acoustic signals 250 are transmitted along a transmission direction 255 with a vector component parallel to the direction of fluid flow 650. In alternative embodiments the array of transmitter elements 200 is oriented along a fixed axis that is not parallel to the long dimension of the fluid vessel 600 being monitored or oriented along an adjustable axis. In an example embodiment, the system comprises 16-32 CMUT transmitter elements operable within a range of interest of approximately 5-8 MHz as shown in FIG. 5, but in alternative embodiments, any number of transmitter elements, of any suitable transducer types operating within a different frequency range of interest may be used.

The array of receiver elements 300 functions to receive acoustic echo signals 250 originating from the array of transmitter elements 200, each receiver element in the array 300 being capable of providing an output signal based on received acoustic signals 250. In one embodiment, the array of receiver elements 300 comprises piezoelectric transducer elements, wherein reflected and received mechanical vibrations are converted to electrical energy by the piezoelectric receiver elements and analyzed. In another embodiment, the array of receiver elements 300 comprises capacitive micromachined ultrasonic transducer (CMUT) elements, which generate signals in response to capacitance changes upon receiving incident acoustic signals.

In the embodiment of the array of receiver elements 300 that includes piezoelectric receiver elements, individual receiver elements are coupled together to form an array that functions to receive acoustic signals 250 originating from the array of transmitter elements 200. Incident acoustic signals cause deformations of the piezoelectric receiver material, which generates an electric signal that can be measured and analyzed to determine properties of an object (e.g. blood flow in a blood vessel) reflecting the acoustic signals. The receiver material may be natural or synthetic. In one embodiment, the piezoelectric receiver elements are physically distinct from the piezoelectric transmitter elements (e.g. transit-time or transmission ultrasound systems), which can be used to accomplish continuous wave measurements. In another embodiment, each piezoelectric element can function as both a transmitter and a receiver, which can be used to accomplish pulsed wave measurements (e.g. Doppler ultrasound systems).

In the embodiment of the array of receiver elements 300 that includes CMUT receiver elements, as shown in FIG. 4, each receiver element is defined by wiring together electrodes on individual CMUT units 150. The array of receiver elements 300 is then composed of multiple receiver elements. Incident acoustic waves are detected by the receivers using capacitive detection, which involves modulations in CMUT capacitance and is observed as modulations in the distances between the plates of CMUT units 150. The capacitance modulations result in current flow in an electronics system 400, as shown in FIG. 5, which can be amplified or conditioned for further processing. Preferably, the receiver elements 300 are composed of an insulating material, such as a dielectric material, coupled to a metal electrode. Alternatively, the plates are entirely composed of a conductive material or a semiconductor.

In the preferred embodiment, the array of receiver elements 300 is arranged approximately orthogonal to the array of transmitter elements 200 to facilitate receiving acoustic signals 250 with an adjustable acoustic aperture 320. In alternative embodiments, however, the array of receiver elements 300 can be arranged along a non-orthogonal axis in relation to the array of transmitter elements 200. In an example embodiment using CMUT elements, the CMUT device comprises 128 receiver elements operable within a range of interest of approximately 5-8 MHz as shown in FIG. 5, but in alternative embodiments, any number of receiver elements operating within a different frequency range of interest may be used, in order to increase or decrease the potential range of acoustic aperture 320 characteristics that can be achieved.

In the preferred embodiment, each transmitter or receiver element in the arrays of transmitter and receiver elements 200, 300 is preferably part of an integrated circuit/transducer device. In the preferred embodiment, the transmitter and receiver elements in an integrated circuit/transducer device may be similar to that described in U.S. Patent Publication No. 2007/0167811 or U.S. Pat. No. 7,888,709, which are both incorporated in their entirety by this reference. In a variation of the preferred embodiment, the arrays of transmitter and receiver elements 200, 300 may be a single array, wherein the system further comprises transmit-receive (T/R) switches that are configured to switch elements between transmitting and receiving functions.

As described above, the arrays of transmitter and receiver elements 200, 300 are preferably positioned relative to a blood vessel 700 being monitored such that the transmitter elements are arranged serially in a direction approximately parallel to the flow direction 650 in the monitored blood vessel 700, which allows the acoustic signal transmission direction 255 to be steered by modifying a vector component of the signal along the flow direction 650. In this preferred embodiment, the array of receiver elements 300 are thereby arranged approximately orthogonal to the blood flow direction 650 and to the array of transmitter elements 200. In an example embodiment using CMUT elements, this arrangement can be achieved with the array of transmitter elements 200 defined by wiring together electrodes located on the lower plates of individual CMUT units 150, and with the array of receiver elements 300 defined by wiring together electrodes located on the upper plates of individual CMUT units 150. However, in a variation of the CMUT embodiment, the arrays of transmitter and receiver elements 200, 300 may alternatively be oriented in any suitable manner using any suitable wiring configurations. In a preferred embodiment, the arrays of transmitter and receiver elements 200, 300 form a rectangular array approximately 1.25 mm wide and 20 mm long, as shown in FIG. 1B. However, the system may include arrays of any suitable numbers of elements, size, and/or geometry, according to desired acoustic signal transmission characteristics and/or desired acoustic aperture characteristics.

The electronics system 400 functions to configure the arrays of transmitter and receiver elements 200, 300 to adjust acoustic signal transmission and receiving parameters. The electronics system 400 can also function to convert output signals provided by receiver elements into voltages, combine the output signals, which have been converted into voltages, and condition at least one combined output signal 350. The electronics system 400 preferably comprises at least analog electronics, including complementary metal-oxide-semiconductor (CMOS) elements, but can further comprise digital electronics with analog-to-digital converters (ADCs). In the preferred embodiment, the electronics system 400 is monolithically integrated with the transmitter and receiver elements in a single-chip configuration. In one variation of the preferred embodiment the array of transmitter elements 200 and the array of receiver elements 300 comprise CMUT elements and are integrated with the electronics system 400 using a single-chip configuration. In alternative embodiments, the electronics system 400 and the transmitter and receiver elements may be located on separate chips, such that the arrays of transmitter and receiver elements 200, 300 and the electronics system 400 are not integrated using a single-chip configuration.

In the preferred embodiment, the electronics system 400 comprises a transmitter control subsystem 410, which functions to control the characteristics of the acoustic signals transmitted by the array of transmitter elements 200. In the preferred embodiment the transmitter control subsystem 410 is configured to control the transmission direction 255 of acoustic signals 250 emitted by the array of transmitter elements 200, but in alternative embodiments the transmitter control subsystem 410 can be further configured to adjust the intensity, beam dimensions, beam location, or any other suitable parameter of acoustic signals 250 emitted by the array of transmitter elements 200.

In the preferred embodiment, the transmitter control subsystem 410 is configured to steer the acoustic signals 250 along an adjustable transmission direction 255. This can be achieved with an electronics system 400 capable of implementing adaptive acoustic signal steering techniques, or alternatively conventional acoustic signal steering techniques, to control the phase and amplitude of an acoustic signal emitted by each transmitter element—interfering signals emitted by the transmitter elements undergo constructive and/or destructive interference in a manner directed by the transmitter control system 410, resulting in transmission of acoustic signals 250 along a controlled direction. The adaptive acoustic signal steering techniques preferably use received acoustic echo signals 250 originating from the transmitter elements to redirect the acoustic signal transmission direction 255 as, for example, to alter the angle of attack of transmitted acoustic signals. In the preferred embodiment the transmission direction 255 is related to a flow direction 650 in a blood vessel 700 being monitored by an insonification angle 260, which can be varied between approximately 10 and 80 degrees. In one embodiment the transmitter control subsystem 410 is configured to steer acoustic signals 250 within a two-dimensional plane, and in an alternative embodiment the transmitter control subsystem 410 is configured to steer acoustic signals 250 within a three-dimensional space. In yet another embodiment, the transmitter control subsystem 410 is configured to steer separate sets of acoustic signals 250 in multiple directions and within multiple planes, using different portions of the array of transmitter elements 200.

In one embodiment the transmitter control subsystem 410 includes a pulser board that controls the acoustic signal pulses being emitted by the transmitter elements, by implementing an acoustic signal steering technique as described above. In an example embodiment, the pulser board has 32 elements corresponding to respective transmitter elements, as shown in FIG. 5. Preferably, each transmitter element in the array of transmitter elements is related to a corresponding pulser board element in a one-to-one configuration, but alternatively multiple transmitter elements can be related a pulser board element in a many-to-one configuration.

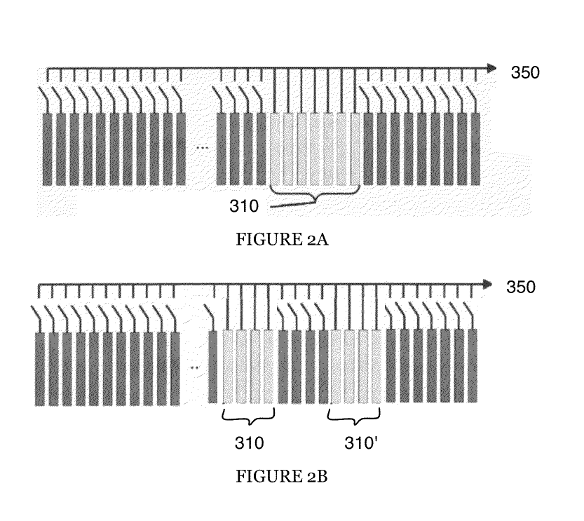

The electronics system 400 of the preferred embodiment also comprises a receiver control system 420, which functions to selectively activate and deactivate receiver elements in the array of receiver elements 300, thus defining an acoustic aperture characterized by activated receiver elements 310. As shown in FIG. 2A, a selected portion of the array of receiver elements 300 may be activated or connected to communicate their electrical outputs to one or more signal output lines, such that the activated receiver elements 310 define an acoustic aperture 320. The unselected or inactivated/deactivated receiver elements may remain unconnected to the signal output lines, as shown in FIG. 2A. Activating a selected portion of the receiver elements may be performed by techniques known to one ordinarily skilled in the art, or by any suitable means. For example, transmitter/receiver switches may be controlled by the output of a serial shift register integrated on the same substrate as the transducer elements (the integrated circuit/transducer device). The size of the acoustic aperture 320 may then be adjusted by the receiver control system 420 by changing the number of activated receiver elements 310, which is achieved in the preferred embodiment by implementing a serial shift register. Similarly, the position of the acoustic aperture 320 relative to the object being monitored may be adjusted by changing the location of activated receiver elements 310, particularly by adjusting which receiver elements in the array 300 are activated, which is achieved using the serial shift register in the preferred embodiment.

The receiver control subsystem 420 preferably provides a digital control scheme over a beam of received acoustic signals, by electronically adjusting and moving the acoustic aperture 320. This digital control scheme enables customization of the device in response to the needs of the particular device application (e.g. adjustment to a particular fluid vessel size or depth). Furthermore, the digital control scheme enables precise placement of the acoustic aperture 320 over a region of interest, with less dependence on accurate physical placement of the receiver array 300. In particular, precise placement of the acoustic aperture 320 over the region of interest maximizes the signal contribution from that region, while minimizing undesirable or irrelevant contributions from other regions. Provision of a digital control scheme facilitates the tracking aspects of the system 100, which can be used in a method for unattended monitoring of blood flow, as described below. As shown in FIG. 2A, in some embodiments, the receiver control subsystem 420 may selectively activate multiple portions of the receiver elements, such as to define multiple acoustic apertures 320, 320′ with variable sizes and/or locations or a “split” acoustic aperture. For example, the device may include two acoustic apertures 320, 320′ each intended to receive echoed acoustic signals regarding blood flow in one of multiple adjacent blood vessels being monitored. This configuration also enables unattended tracking and monitoring of multiple blood vessels.

In the preferred embodiment of the system 100, the electronics system 400 further comprises an analog adder circuit 430, which functions to combine or sum individual output signals provided by activated receiver elements 310, wherein said output signals have been converted into output voltages. The analog adder circuit 430 thus functions to combine the output signals of the receiver elements into a reduced number of electrical channels for conditioning or processing. The analog adder circuit 430 preferably functions to combine the converted output signals into one combined output 350, or alternatively, a reduced number of combined outputs. In the preferred embodiment, the receiver control subsystem 420 and the analog adder circuit 430 function together to select a subset of receiver elements from the array of receiver elements 300 at a time, scan through different activated receiver element combinations, and output a combined signal 350 that can be used to determine a blood flow parameter for unattended monitoring of blood flow. In an alternative embodiment a reduced number of combined output signals 350′ can be used to produce ultrasound image data.

The electronics system 400 of the preferred embodiment also comprises signal processing circuitry 440, which functions to condition a combined signal 350 provided by the analog adder circuit 430. As shown in FIGS. 1 and 5, the signal processing circuitry 440 preferably comprises a gain amplifier, which functions to amplify obtained acoustic signals 250 and/or a combined signal 350. In an alternative embodiment, the signal processing circuitry 440 can further comprise filters, which function to filter obtained acoustic signals 250 and/or a combined signal 350, demodulators, which function to demodulate an echo signal to provide in-phase and quadrature (I and Q) components indicative of positive and negative flow velocity, and ADCs. The signal processing circuitry can alternatively comprise any suitable elements. Signals processed by the signal processing circuitry 440 may be further passed to other internal on-chip processors and/or external processors 500 such as a personal computer for higher-level processing, analysis, image display, and/or data storage.

In one embodiment, the signal processing circuitry 440 comprises readout electronics that function to process signals received from the activated receiver elements 310. The readout electronics preferably amplify or otherwise condition the signal received from the activated receiver elements using single-stage or multiple-stage CMOS amplifier circuits, or alternatively any suitable circuits capable of conditioning an unconditioned signal. The readout electronics preferably have an input connected to the array of receiver elements 300, whereby the input allows communication of an unconditioned signal to the readout electronics.

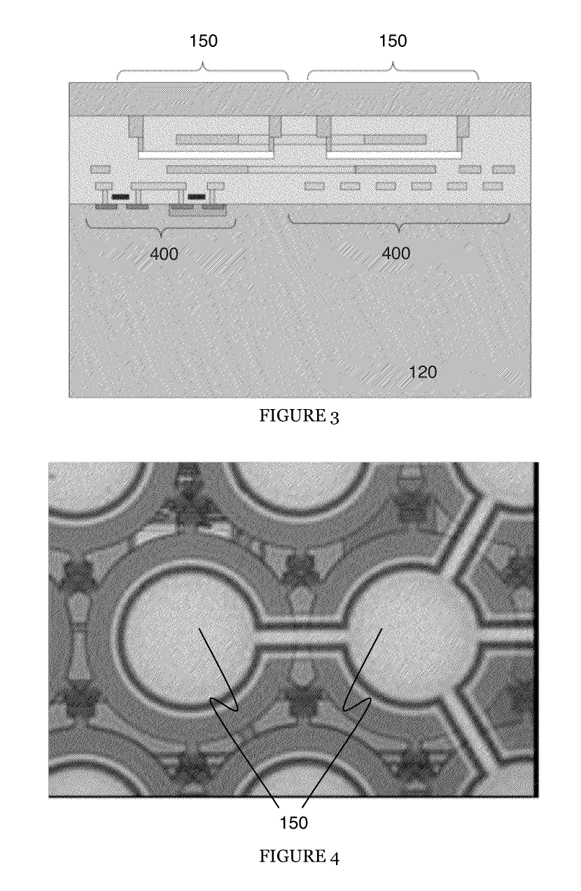

The arrays of transmitter and receiver elements 200, 300 and at least a portion of the electronics system 400 are preferably incorporated in a series of integrated circuit/transducer devices. As shown in FIG. 3, a integrated circuit/transducer device may include a substrate 120, one or more ultrasonic transducers as transmitter and/or receiver elements coupled to the substrate 120, and at least a portion of the electronics system 400 coupled to the substrate. This arrangement, which increases the proximity of the arrays of transmitter and receiver elements 200, 300 to the electronics system 400, allows for a reduction in parasitic signal losses and reduces the physical size of the system. The series of integrated circuit/transducer devices may be incorporated in a handheld probe communicating with a central console in an ultrasound system, similar to that described in U.S. Patent Publication No. 2007/0167811 entitled “Capacitive micromachined ultrasonic transducer” or U.S. Pat. No. 7,888,709 entitled “Capacitive micromachined ultrasonic transducer and manufacturing method”, which are each incorporated in its entirety by this reference. However, alternatively the transmitter and receiver elements 200, 300, and electronics system 400 may alternatively be arranged as separate components (e.g. on separate chips) or in any suitable manner.

The preferred embodiment of the system 100 also comprises a fastener 600 to position the array of transmitter elements, the array of receiver elements, and the electronics system on a patient, wherein the fastener 600 functions to maintain the arrays 200, 300 and the electronics system 400 in a substantially stable position relative to the blood vessel(s) 700 being monitored. In a first variation, as shown in FIG. 5B, the fastener 600 is a housing for the arrays of transmitter and receiver elements 200, 300 and electronics system 400 that comprises an adhesive to attach the housing to a patient. In the first variation the housing may be flexible, or alternatively may be stiff. In a second variation, as shown in FIG. 5C, the fastener 600 is a band configured to house the arrays of transmitter and receiver elements 200, 300 and the electronics system 400, and to be worn by the patient. In a third variation the fastener 600 is a clip that attaches the arrays of transmitter and receiver elements 200, 300 and the electronics system 400 to the patient. In a fourth variation the fastener is a strap that attaches the arrays of transmitter and receiver elements 200, 300 and electronics system 400 to the patient.

The preferred embodiment of the system further comprises a processor 500 that is in communication with the electronics system 400 and functions to provide feedback to the electronics system 400 to enable self-alignment of transmitted acoustic signals based on received acoustic signals, and to determine a blood flow parameter based on Doppler flow information extracted from a combined signal 350. The processor 500 preferably comprises a first module configured to provide feedback to the electronics system and a second module to relate a Doppler flow parameter derived from a combined signal 350 to a parameter based on the transmission direction 255 (e.g. an insonification angle 260). The first module preferably communicates with the transmitter control subsystem 410 and the receiver control subsystem 420, to provide feedback signals to automatically adjust the transmission direction 255 and/or an acoustic aperture 320. This communication facilitates unattended tracking and monitoring of blood flow in a blood vessel 700 being monitored. Information from the first module can also be used to provide feedback to at least one of the analog adder circuit 430 and the signal processing circuitry 440, providing adjustments to signal combination processes and/or signal conditioning processes. The second module is preferably configured to relate a Doppler flow parameter derived from a combined signal 350 to a transmission direction parameter or an acoustic aperture parameter. The Doppler flow parameters of interest preferably include Doppler amplitude and Doppler phase shift, but can alternatively include any appropriate Doppler parameter of interest. Information from the second module is also preferably used in the determination of a blood flow parameter, such as blood flow velocity or blood volume flow rate.

In one embodiment, the system 100 or a portion thereof is preferably configured to perform the method further described below.

### Method for Unattended Monitoring of Blood Flow

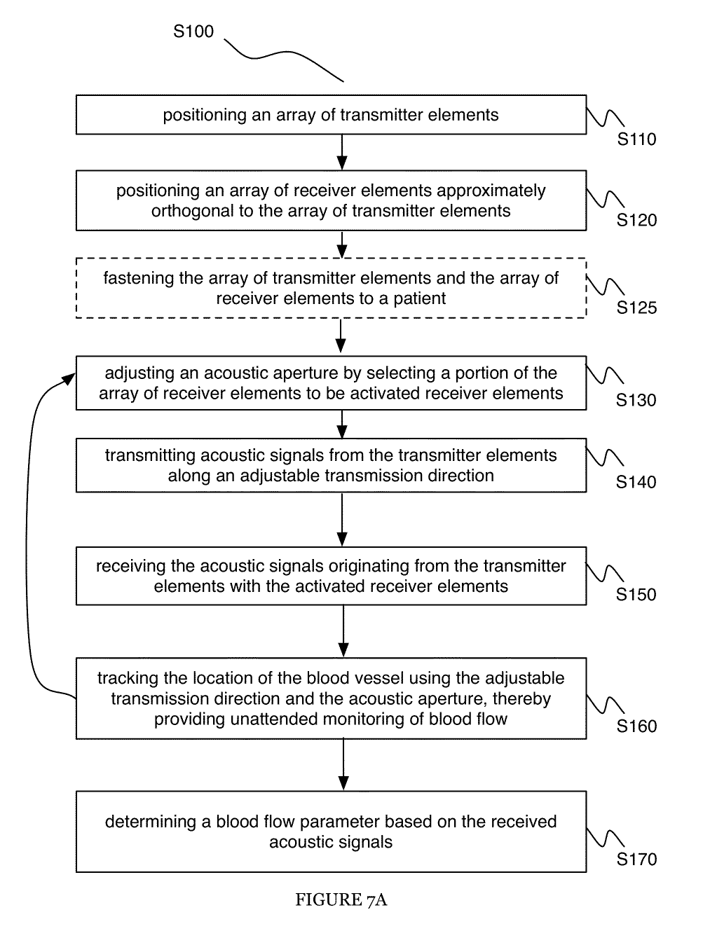

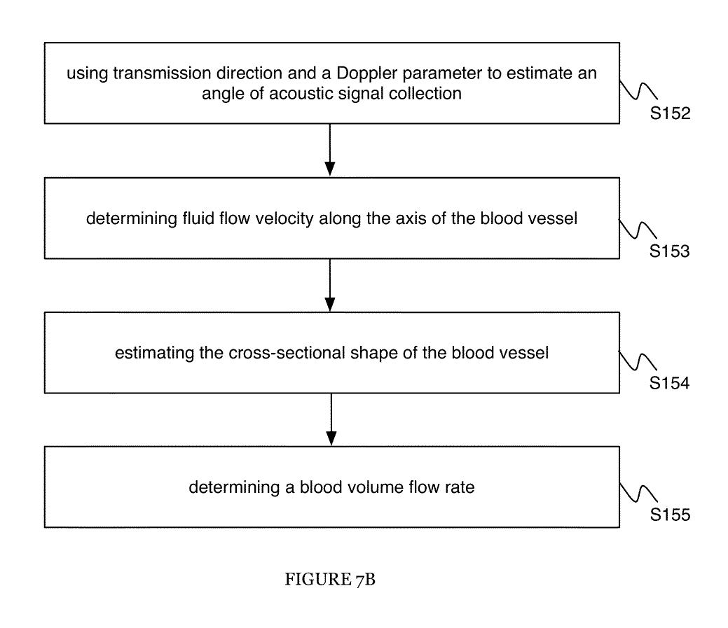

In an embodiment, as shown in FIGS. 6 and 7, the method S100 for unattended monitoring of blood flow passing in a flow direction in a blood vessel comprises positioning an array of transmitter elements along a direction approximately parallel to the flow direction S110, fastening an array of receiver elements approximately orthogonal to the array of transmitter elements S120; adjusting an acoustic aperture by selecting a portion of the array of receiver elements to be activated receiver elements S130; transmitting acoustic signals along an adjustable transmission direction S140; receiving the acoustic signals originating from the transmitter elements with the activated receiver elements S150, wherein the transmission direction is automatically aligned based on the received acoustic signals; tracking the location of the blood vessel using the adjustable transmission direction and the acoustic aperture, thereby providing unattended monitoring of blood flow S160; and determining a blood flow parameter based on the received acoustic signals S170. The step of transmitting acoustic signals S140 also includes adjusting the transmission direction 255 by modifying a vector component of the transmitted acoustic signals along the flow direction 650. Similar to the system 100 described above, the method can be used to monitor blood flow such as in blood vessels in a human or animal. In one variation, the method may be used to determine volume flow rate through the blood vessel, without requiring an image of the blood vessel.

Positioning an array of transmitter elements, such that the transmitter elements are arranged serially along a direction approximately parallel to a blood flow direction S110 functions to facilitate steering of a vector component of a transmitted acoustic signal that is along the flow direction, substantially aligning a vector component of the transmitted acoustic signals with the flow direction. Positioning the array substantially over a blood vessel or network of blood vessels to be monitored facilitates unattended monitoring of blood flow, such that the system 100 can more efficiently track a blood vessel. Preferably, the array of transmitter elements is positioned in a location to provide unattended detection of variations in blood flow. In one embodiment, the array of acoustic transmitter elements can be positioned between dialysis needle points, in order to provide unattended monitoring of blood flow in a patient undergoing hemodialysis. In another embodiment, the array of acoustic transmitter elements can be positioned over a single blood vessel or vascular network of interest, for instance, to enable detection of thromboses or thromboembolisms.

Positioning an array of acoustic receiver elements approximately orthogonal to the array of transmitter elements S120, functions to provide a useful range of potential acoustic apertures to receive reflected acoustic signals. In the preferred embodiment, the orthogonal orientation of the arrays of transmitter and receiver elements maximizes the range of acoustic apertures that can be achieved for a given number of receiver elements in the array of acoustic receiver elements. In one embodiment, positioning the arrays of acoustic transmitter and receiver elements S110, S120 comprises using a fastener to enable unattended monitoring of blood flow, wherein the fastener is a housing comprising an adhesive layer. In another embodiment, the fastener is a band configured to be worn by a patient. Other variations of the fastener are detailed above in the description of the system 100.

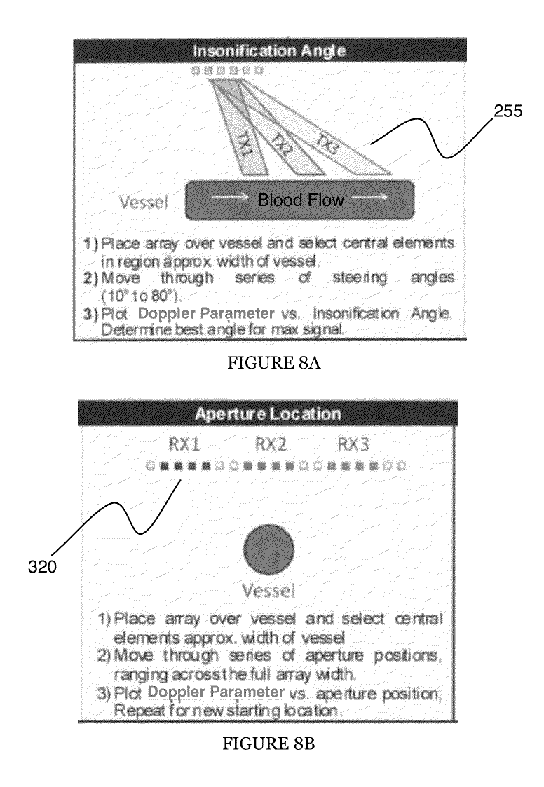

Adjusting an acoustic aperture by selecting a portion of the array of receiver elements to be activated receiver elements S130 functions to maximize signal contribution from a particular region of interest while minimizing signal contribution from regions not of interest, thereby enhancing accuracy and precision of blood flow monitoring. Adjusting an acoustic aperture 320 preferably includes selecting a portion of the array of receiver elements 300 to be activated receiver elements 310, deactivating receiver elements not in the selected portion of the array of receiver elements 300, and may further include enabling output signals of activated receiver elements 310 to be combined for further conditioning and/or processing. In one embodiment, the acoustic aperture may be defined such that the activated receiver elements 310 span the approximate width of a blood vessel 700 being monitored, by defining the number and/or position of activated receiver elements 310. The step of defining an acoustic aperture S120 may be performed in close correlation with (e.g., immediately prior to, during, or possibly immediately following) the step of transmitting acoustic signals S140. The acoustic aperture 320 may be repeatedly defined and/or adjusted as needed, in coordination with adjustments to the transmission direction 255. For example, as shown in FIG. 8B, the location of the acoustic aperture may be adjusted along a range of positions until an optimal or other desired position of acoustic aperture 320 is identified, by relating the acoustic aperture 320 to a Doppler parameter (e.g. integrated power of the Doppler signal, or magnitude of the Doppler frequency shift) based on acoustic signals received by the activated receiver elements 310. Similarly, as shown in FIG. 8C, the width of the acoustic aperture 320 may be adjusted along a range of widths, such as a single receiver element to the full array of the receiver elements 300, until an optimal or other desired width of acoustic aperture is identified, by relating the acoustic aperture 320 to a Doppler parameter (e.g. integrated power of the Doppler signal, or magnitude of the Doppler frequency shift) based on acoustic signals 250 received by the activated receiver elements 310.

Transmitting acoustic signals from the transmitter elements along an adjustable transmission direction S140 functions to transmit a beam of acoustic signals 250 along an adjustable transmission direction 255 without physically moving the transmitting elements. This can be achieved in one embodiment by using a pulser board to control sequences of acoustic signals emitted by transmitter elements. By using an array of transmitter elements, the time and distance between acoustic signals emitted by individual transmitter elements can be controlled by the pulser board, resulting in an acoustic signal beam with an adjustable transmission direction 255. As shown in FIG. 8A, the angle between the transmission direction and the blood vessel being monitored (the insonification angle) can be adjusted over a range of angles, such as 10°-80° (an angular displacement of 70°) but in alternative embodiments, a reduced or enlarged range of angles may be used. Transmitting acoustic signals along a transmission direction 255 and adjusting the transmission direction 255 can then be used to relate an insonification angle 260 to a Doppler parameter based on acoustic signals originating from the transmitter elements 200 and received by an array of receiver elements 300. In the preferred embodiment, the insonification angle 260 can be adjusted until the optimal angle for receiving a maximum acoustic signal is identified, such as on a plot of a relevant Doppler parameter vs. insonification angle (FIG. 8A), wherein the relevant Doppler parameter is a maximum frequency shift, signal power, or a combination of the two. Receiving a maximum acoustic signal ultimately increases data quality by reducing adverse effects due to noise, loss, and other factors. In alternative embodiments other Doppler parameters, or combinations thereof, may be related to other parameters of the transmission direction 255, in determining an appropriate transmission direction 255.

Receiving the acoustic signals originating from the transmitter elements with the activated receiver elements S150, functions to obtain transmitted and reflected acoustic signals originating from the array of transmitter elements 200. In the preferred embodiment, the activated receiver elements 310, which define an acoustic aperture 320, receive the acoustic signals 250 for further conditioning. In alternative embodiments, multiple portions of activated receiver elements may receive the acoustic signals for further conditioning, as in a situation with multiple or split acoustic apertures 320, 320′.

Tracking the location of the blood vessel using the adjustable transmission direction and the acoustic aperture, thereby providing unattended monitoring of blood flow S160, functions to automatically maintain a given level of an acoustic parameter characterizing acoustic signals reflected from blood vessel 700 being monitored, which enables unattended monitoring of blood flow. In the preferred embodiment the transmission direction is automatically aligned based on the received acoustic signals, in order to maintain a given level of an acoustic signal parameter (e.g. Doppler parameter) characterizing acoustic signals received by the activated receiver elements. For example, a Doppler parameter (e.g. Doppler power) is maintained at a given level by automatically aligning the transmission direction, to enable tracking. In one variation, the Doppler parameter is maintained at a maximum level (the level at which the parameter peaks and plateaus) by adjusting the transmission direction. In the preferred embodiment, tracking the location of the blood vessel also comprises adjusting an acoustic aperture to maintain a given level of an acoustic signal parameter (e.g. Doppler parameter) characterizing acoustic signals received by the activated receiver elements. In the preferred embodiment, adjusting the acoustic aperture comprises adjusting the acoustic aperture size and/or position in order to maintain a given level of an acoustic signal parameter characterizing acoustic signals received by the activated receiver elements. In one variation of tracking the location of the blood vessel S160, the adjustable transmission direction may initially be set to a given value, and the acoustic aperture size may be held constant while the acoustic aperture position is adjusted S130 to determine a location of the blood vessel being monitored. The acoustic aperture size can subsequently be adjusted to determine a dimension of the blood vessel being monitored. Then, transmitting acoustic signals along an adjustable transmission direction S140 can be performed in an alternating fashion with receiving acoustic signals S150, in parallel with adjusting the adjustable transmission direction to achieve a maximum level of a Doppler parameter (e.g. Doppler power) characterizing acoustic signals received by activated receiver elements. Tracking the location of the blood vessel S160 can then be performed by adjusting the adjustable transmission direction and the acoustic aperture size by activating and deactivating receiver elements to maintain the maximum level of the Doppler parameter.

In another variation of the preferred embodiment, adjusting an acoustic aperture S130 size and position, by activating and deactivating receiver elements, can be performed substantially in parallel with transmitting acoustic signals along an adjustable transmission direction S140 and receiving acoustic signals S150, wherein S140 and S150 are performed in an alternating fashion in order to initially locate and determine a dimension of a blood vessel to be monitored using the acoustic aperture. Once the blood vessel has been located and the dimension has been determined, the adjustable transmission direction can be further adjusted to achieve a maximum level of a Doppler parameter (e.g. Doppler amplitude) characterizing acoustic signals received by the activated receiver elements. This maximum level can then be maintained by adjusting the transmission direction and/or acoustic aperture, in order to enable tracking the location of the blood vessel S160. The method S100 may also comprise tracking the location of a second blood vessel for unattended monitoring of blood flow in multiple blood vessels.

In the preferred embodiment, tracking the location of the blood vessel S160 is performed using a processor 500 in communication with an electronics system 400, wherein the processor provides feedback based on variations in acoustic signal parameters to the electronics system, which comprises transmitter and receiver control subsystems 410, 420.

The method S100 may further comprise converting the received acoustic signals and combining the converted signals S165, which functions to achieve a reduced number of outputs for analysis and/or processing. In one embodiment, converting the received acoustic signals comprises converting the received acoustic signals 250 into electronic signals using piezoelectric properties of the transducer materials, and in another embodiment, converting the received acoustic signals comprises converting the received acoustic signals into electronic signals using capacitance changes of CMUT transducer components. In either embodiment, combining the converted received acoustic signals into a reduced number of signal outputs can be performed by using an analog adder circuit 430, which is detailed in the above description of a system for unattended monitoring of blood flow.

Determining a blood flow parameter based on the received acoustic signals S170 functions to determine an appropriate metric that can be used to monitor variations in blood flow. In the preferred embodiment, acoustic signals received by the activated receiver elements are analyzed and processed to determine a blood flow velocity using methods known by one ordinarily skilled in the art or any suitable analytical method. The blood flow velocity can be used to subsequently determine an additional blood flow parameter, such as blood volume flow rate, as a metric for use in unattended blood flow monitoring.

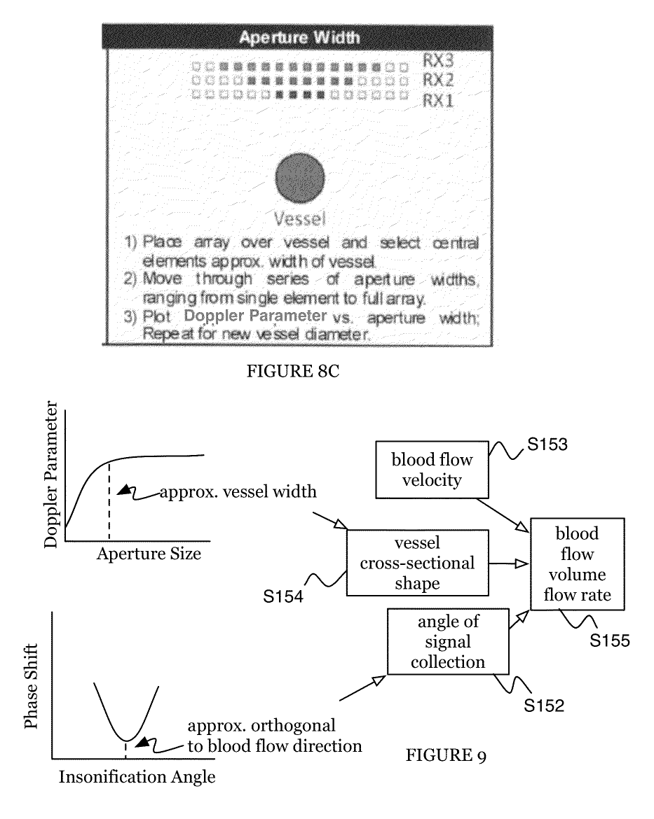

As shown in FIGS. 7-9, determining a blood volume flow rate comprises using a blood flow velocity, a blood vessel dimension (determined using the acoustic aperture), and an angle of acoustic signal transmission/collection (determined using the adjustable transmission direction). In the preferred embodiment, determining a blood volume flow rate is performed without requiring acquisition of an acoustic image (e.g. ultrasonic image or series of images); however, in alternative embodiments, determining a blood volume flow rate can involve the acquisition of an image derived from acoustic signal data, in order to determine blood vessel dimensions. Processing the acoustic signals includes using the transmission direction and a Doppler parameter of the received acoustic signals to estimate an angle of acoustic signal collection S152, determining blood flow velocity along the axis of the fluid vessel S153, estimating the cross-sectional shape of the blood vessel from the acoustic aperture and a Doppler parameter of the received acoustic signals S154, and determining a blood volume flow rate from the blood flow velocity and the cross-sectional shape S155.

Using the transmission direction and a Doppler parameter of the received acoustic signals to estimate an angle of acoustic signal collection S152 functions to enable determination of a blood flow velocity along the axis of a blood vessel being monitored. This step preferably includes steering the transmitted acoustic signals through a range of prescribed insonification angles 260 (by adjusting the transmission direction 255) and monitoring the Doppler phase shift of the signals corresponding to the insonification angle. The angle at which minimum phase shift is measured, which occurs when the transmitted acoustic signals are orthogonal to the direction of fluid flow, may be considered to correspond to a “zero” angle. The transmission direction 255 and/or angle at which signals are received by the receiver elements (related by conservation laws) may be steered to a known angle relative to the “zero” angle, and therefore to a known angle relative to the blood flow direction 650. Preferably, the transmitted acoustic signals are initially aimed at any suitable angle (e.g. on either extreme end of a possible range of angles, or at an estimated initial angle) and swept in one or more directions. In a first variation, the transmitted acoustic signals 250 may pass in one direction from an extreme insonification angle 260 to another extreme insonification angle 260 of a range (e.g. 10°-80°, or 80°-10°) until the minimum phase shift is determined to determine the “zero” angle. In a second variation, the transmitted acoustic signals may be aimed at various sampled points in a range such as to converge on the insonification angle 260 at which minimum phase shift is determined. However, any suitable method may be used to estimate the angle of acoustic signal collection.

Preferably, determining blood flow velocity along the axis of the blood vessel S153 includes analyzing the combined output signal to calculate a projected blood flow velocity based on at least one Doppler parameter of the received acoustic signals, which is known by one ordinarily skilled in the art. The projected blood flow velocity can then be used, with the angle of acoustic signal collection, to determine the blood flow velocity along the axis of the blood vessel.

Estimating the cross-sectional shape of the blood vessel S154 preferably includes incrementally changing the size of the acoustic aperture 320 and monitoring the strength of the received acoustic signals (e.g. Doppler power) corresponding to the acoustic aperture size. The size of acoustic aperture 320 when increasing the acoustic aperture 320 no longer increases the strength of the received acoustic signals scales with the dimension defining the cross-sectional shape of the blood vessel (e.g., blood vessel width, which can be used to determine blood vessel cross-sectional area based on cross-section geometric assumptions). In a first variation, an initial acoustic aperture 320 includes a single receiver element (or a relatively small number of active receiver elements) and the strength of the received acoustic signals 250 is monitored as the acoustic aperture 320 is increased, until the strength of the received acoustic signals plateaus. In a second variation, an initial acoustic aperture 320 includes a full array of activated receiver elements 310 (or a relatively large number of activated receiver elements 310), and the strength of the received acoustic signals 250 is monitored as the acoustic aperture 320 is reduced until the strength of the received acoustic signals 250 begins to drop. In both of these variations, the final size of the acoustic aperture 320 or number of activated receiver elements 310 scales with an estimated width of the blood vessel. In a third variation, an initial acoustic aperture 320 may include any suitable estimate of the blood vessel width (e.g., based on operator experience, prior knowledge, or intuition) and be incrementally increased and/or reduced while monitoring the combined output signal strength, similar to the first and second variations of this estimating step. However, any suitable method may be used to estimate the cross-sectional shape of the blood vessel.

Determining a blood volume flow rate from the blood flow velocity and the cross-sectional shape S155 functions to determine a quantifiable blood flow parameter that can be used as a metric in unattended monitoring of blood flow. After blood flow velocity, blood vessel cross-sectional shape, and angle of acoustic signal collection (and/or angle of acoustic signal transmission, or insonification) measured or estimated by the above-described steps, these measurements and/or estimations may be used to calculate blood volume flow rate, defined as the produce of the blood flow velocity along the axis of the blood vessel, and the cross-sectional area of the blood vessel. In alternative embodiments other suitable metrics of the blood flow can be determined as is known by one ordinarily skilled in the art.

The method of the preferred embodiment and variations thereof can be embodied and/or implemented using at least in part a machine configured to receive a computer-readable medium storing computer-readable instructions. The instructions are preferably executed by computer-executable components preferably integrated with the system and one or more portions of the processor 140 and/or the controller 150. The computer-readable medium can be stored on any suitable computer-readable media such as RAMs, ROMs, flash memory, EEPROMs, optical devices (CD or DVD), hard drives, floppy drives, or any suitable device. The computer-executable component is preferably a general or application specific processor, but any suitable dedicated hardware or hardware/firmware combination device can alternatively or additionally execute the instructions.

The FIGURES illustrate the architecture, functionality and operation of possible implementations of systems, methods and computer program products according to preferred embodiments, example configurations, and variations thereof. In this regard, each block in the flowchart or block diagrams may represent a module, segment, or portion of code, which comprises one or more executable instructions for implementing the specified logical function(s). It should also be noted that, in some alternative implementations, the functions noted in the block can occur out of the order noted in the FIGURES. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

As a person skilled in the art will recognize from the previous detailed description and from the figures and claims, modifications and changes can be made to the preferred embodiments of the invention without departing from the scope of this invention defined in the following claims.

We claim:

1. A system for unattended monitoring of blood flow in a blood vessel comprising:
an array of transmitter elements configured to transmit acoustic signals along a transmission direction;
an array of receiver elements configured to receive acoustic signals originating from the array of transmitter elements,
wherein the array of transmitter elements is arranged approximately orthogonal to the array of receiver elements, and
wherein each receiver element is configured to provide an output signal in response to received acoustic signals;
an electronics system comprising:
a transmitter control subsystem configured to adjust the transmission direction,
a receiver control subsystem configured to selectively activate and deactivate receiver elements, thus defining an acoustic aperture characterized by activated receiver elements,
signal processing circuitry configured to condition at least one output signal; and
a processor in communication with the electronics system and configured to:
provide feedback to the electronics system to enable self-alignment of transmitted acoustic signals based on received acoustic signals, thereby providing unattended monitoring of blood flow, and
determine a blood flow parameter based on an acoustic parameter extracted from the received acoustic signals.

2. The system of claim 1, wherein the array of transmitter elements and the array of receiver elements are distinct arrays.

3. The system of claim 1, further comprising a transmit-receive (TR) switch to switch between transmitting and receiving functions.

4. The system of claim 1, wherein at least one of the array of transmitter elements and the array of receiver elements comprises piezoelectric elements.

5. The system of claim 1, wherein at least one of the array of transmitter elements and the array of receiver elements comprises capacitive micromachined ultrasonic transducer (CMUT) elements.

6. The system of claim 5, wherein the array of transmitter elements comprises a first set of plates of a set of CMUT units and the array of receiver elements comprises a second set of plates of the CMUT units.

7. The system of claim 5, wherein at least two of the array of transmitter elements, the array of receiver elements, and the electronics system are integrated on a single chip.

8. The system of claim 1, wherein the electronics system comprises complementary metal-oxide-semiconductor elements.

9. The system of claim 1, wherein the electronics system further comprises an analog adder circuit configured to produce a combined output by combining output signals provided by the activated receiver elements,

10. The system of claim 1, wherein the transmitter control subsystem comprises a pulser board configured to control acoustic signal pulses emitted by each transmitter element.

11. The system of claim 10, wherein the transmitter control subsystem is configured to adjust the transmission direction by using a sequence of acoustic signal pulses emitted by individual transmitter elements, controlled by the pulser board.

12. The system of claim 10, wherein each transmitter element in the array of transmitter elements corresponds to a pulser board element in a one-to-one configuration.

13. The system of claim 1, wherein the transmitter control subsystem is configured to adjust the transmission direction in three dimensional space.

14. The system of claim 1, wherein the transmitter control subsystem is configured to adjust the transmission direction over an angular displacement of at least 70 degrees.

15. The system of claim 1, wherein defining an acoustic aperture comprises defining an acoustic aperture size.

16. The system of claim 1, wherein defining an acoustic aperture comprises defining an acoustic aperture position.

17. The system of claim 1, wherein the receiver control subsystem is further configured to selectively activate and deactivate receiver elements, thus defining multiple acoustic apertures.

18. The system of claim 1, wherein the signal processing circuitry is further configured to condition the combined output by filtering at least one output signal.

19. The system of claim 1, wherein the signal processing circuitry is further configured to condition the combined output by amplifying at least one output signal.

20. The system of claim 1, wherein the signal processing circuitry comprises a readout electronics subsystem.

21. The system of claim 1, further comprising a fastener configured to maintain the position the array of transmitter elements, the array of receiver elements, and the electronics system on a patient.

22. The system of claim 21, wherein the fastener is a patch that comprises a housing for at least two of the array of transmitter elements, the array of receiver elements, and the electronics system.

23. The system of claim 22, wherein the patch comprises an adhesive surface.

24. The system of claim 21, wherein the fastener is a band that houses at least two of the array of transmitter elements, the array of receiver elements, and the electronics system.

25. The system of claim 1, wherein the processor comprises a module configured to relate the acoustic parameter to a parameter based on the transmission direction.

26. The system of claim 25, wherein the parameter based on the transmission direction is an insonification angle.

27. The system of claim 25, wherein the module is further configured to relate the acoustic parameter to at least one of an acoustic aperture size and position.

28. The system of claim 25, wherein the processor is configured to determine blood flow velocity.

29. The system of claim 25, wherein the processor is configured to determine blood volume flow rate.

30. The system of claim 29, wherein the processor is configured to determine blood volume flow rate using an image of a blood vessel.

31. The system of claim 1, wherein the processor further comprises a self-checking module to support automatic alignment of the transmitted acoustic signals toward a region of interest.

32. A method for unattended monitoring of blood flow in a blood vessel comprising:
positioning an array of acoustic transmitter elements such that the transmitter elements are arranged serially along a direction approximately parallel to a blood flow direction;
positioning an array of acoustic receiver elements approximately orthogonal to the array of transmitter elements;
adjusting an acoustic aperture by selecting a portion of the array of receiver elements to be activated receiver elements;
transmitting acoustic signals from the transmitter elements along an adjustable transmission direction;
receiving the acoustic signals as echo signals originating from the transmitter elements with the activated receiver elements,
wherein the acoustic aperture is automatically adjusted based on the echo signals received by the activated receiver elements; and
determining a blood flow parameter based on the received acoustic signals.

33. The method of claim 32, further comprising adjusting multiple acoustic apertures by selecting multiple portions of the array of receiver elements to be activated receiver elements.

34. The method of claim 32, further comprising fastening the array of acoustic transmitter elements and the array of acoustic receiver elements to a patient.

35. The method of claim 32, wherein the adjustable transmission direction can be adjusted over an angular displacement of at least 70 degrees.

36. The method of claim 32, wherein the adjustable transmission direction is automatically aligned in order to maintain a given level of an acoustic parameter characterizing acoustic signals received by the activated receiver elements.

37. The method of claim 32, wherein the given level is a maximum level of an acoustic parameter.

38. The method of claim 32, further comprising tracking the location of the blood vessel using the acoustic aperture, thereby providing unattended monitoring of blood flow.

39. The method of claim 38, wherein tracking the location of the blood vessel comprises adjusting the position of the acoustic aperture to maintain a given level of an acoustic parameter characterizing acoustic signals received by the activated receiver elements.

40. The method of claim 38, wherein tracking the location of the blood vessel comprises adjusting the size of the acoustic aperture to maintain a given level of an acoustic parameter characterizing acoustic signals received by the activated receiver elements.

41. The method of claim 40, wherein the given level is a maximum level of a Doppler parameter.

42. The method of claim 38, wherein the size of the acoustic aperture remains substantially constant during tracking.

43. The method of claim 38, further comprising tracking the location of a second blood vessel.

44. The method of claim 32, wherein determining a blood flow parameter comprises determining a blood flow velocity.

45. The method of claim 44, wherein determining a blood flow velocity comprises using a Doppler parameter of the acoustic signals received by the activated receiver elements.

46. The method of claim 45, wherein the Doppler parameter is an integrated power of the Doppler signal.

47. The method of claim 32, wherein determining a blood flow parameter comprises determining a blood volume flow rate.

48. The method of claim 47, wherein determining a blood volume flow rate comprises determining a blood flow velocity and a blood vessel width.

49. The method of claim 48, wherein determining a blood vessel width comprises determining a blood vessel width from the acoustic aperture.

50. The method of claim 32, wherein determining a blood flow parameter comprises determining a blood flow parameter by using an image derived from acoustic signal data.

51. The method of claim 32, wherein determining a blood flow parameter comprises determining an insonification angle by adjusting the adjustable transmission direction and monitoring an acoustic parameter characterizing acoustic signals received by the activated receiver elements.

52. The method of claim 51, wherein the acoustic parameter is a frequency shift.

53. The method of claim 52, wherein determining an insonification angle comprises determining a zero angle, corresponding to a minimum frequency shift.

54. The method of claim 53, wherein determining an insonification angle comprises adjusting the transmission direction between the zero angle and a known insonification angle.
